(19) United States
(12) Patent Application Publication
(10) Pub. No.: US 2024/0024588 A1
(43) Pub. Date: Jan. 25, 2024

(54) AUTOMATIC NEEDLE INSERTER FOR PEN INJECTION SYSTEM

(71) Applicant: BIOCORP PRODUCTION S.A., Issoire (FR)

(72) Inventor: Alain MARCOZ, Issoire (FR)

(21) Appl. No.: 18/256,921

(22) PCT Filed: Dec. 10, 2020
(86) PCT No.: PCT/IB2020/001075

§ 371 Date: Jun. 12, 2023

(51) Int. Cl. A61M 5/32; A61M 5/24

(52) U.S. Cl. CPC A61M 5/3287; A61M 5/24; A61M 2205/0216

(57) ABSTRACT

An automatic needle inserter for a pen injector is provided. The automatic needle inserter has an elongated inserter body dimensioned and configured to receive the pen injector when introduced into a longitudinal bore of elongated inserter body via the proximal extremity thereof. The automatic inserter body is also configured to prevent the pen injection system from exiting the longitudinal bore via the distal extremity of the inserter body. The automatic needle inserter is provided with a rotationally-activated locking means to lock the pen injector in an axial position within the longitudinal bore via rotation of at least a part of the elongated inserter body about the central longitudinal axis from a first, non-locking position to at least one or more second, locking positions.

The present invention relates generally to accessories for injection pen systems.

Injection pen systems are well known per se and are commonly equipped with a proximally located dose setting wheel and injection activator, the dose setting wheel being rotatable about a central longitudinal axis of the pen injection system. The wheel is rotated by the user to select the dose of drug to be administered. The pen is generally configured, either mechanically or electro-mechanically to effect an injection upon activation of an injection activator. Such injection activators are quite commonly a simple press or push-button, in mechanical or electrical contact with the dispensing mechanism located within the pen injection system, the pressing of which causes the injection mechanism to fire and inject the drug contained within the pen injection system. In some pen injector systems, the dose setting wheel is configured to rotate not only during dose setting, but also during injection. This is generally achieved through the inclusion of one or more metallic components, such as a helically wound drive spring located within a housing body of the injection pen system and physically coupled to the dose setting wheel.

Injection pen systems of the type described above are used by both medically trained, and non-medically trained users. Whilst medically trained users are accustomed to manipulating such devices after suitable training, individual non-medically trained users, such as the patients themselves, still sometimes struggle to use these devices correctly, and/or appropriately. Manufacturers of such pen injection systems have furthermore over time attempted to make such systems as easy and foolproof to use as possible.

Notwithstanding the above developments in pen injection system technology, there remains, for some users at least, difficulties in using such pen injection systems correctly. For example, some users that need to administer drugs provided in such injection pen systems also present with neural and/or muscular coordination difficulties, rendering the manipulation of the pen injection systems incomplete, inexact or imprecise. Other users have a fear of either seeing, handling or otherwise manipulating needles, including inserting the needle into the body, and are therefore faced with a significant psychological challenge when attempting to use the most commonly available commercialized injection pen systems, despite the relative improvements in user friendliness. Such challenges can impact a patient's wellbeing and more importantly, observance of the treatment regime involving such pen injection systems that the user-patient of the device is supposed to be following.

As a result, there have been a few attempts to overcome the above difficulties by providing an automatic, or semi-automatic needle inserter. The aim of such needle inserter devices, which can be considered both a separate device and an accessory in their own right, is to facilitate presentation of the needle of the injection pen system, at the correct angle for penetration into the body at the site of the injection, and to do so by allowing the user to locate the pen within the automatic needle inserter, arm the automatic needle inserter so that the injection pen system is ready for injection, and then permit release of the armed injection pen system in a manner which relieves the user from being confronted directly with the sight of having to prick themselves with the needle of the pen injection system.

For example, published U.S. Pat. Nos. 5,980,491A and 6,537,252 B1, both relate to an automatic needle insertion device for a pen-shaped syringe, the device comprising a tubular housing in which an injection pen is mounted in a tubular pen holder, the pen holder being axially movable along a longitudinal axis of the pen and automatic needle insertion device, in a proximal direction, i.e. towards the hands and/or body of a user, to cock a spring which is thereafter released to drive the pen holder with the pen a set distance in a distal direction. The injection pen is connected distally to a drug containing cartridge, and a needle mounted to the distal end of the drug containing cartridge. This system is specific to the products manufactured and commercialized by applicant of these patents.

Additionally, published French patent application FR3079422A1, relates to another automatic needle inserter device, configured to receive an injection pen system, such as an insulin injection pen, the needle inserter device comprising a body with a holder for an injection system, the body being configured to move, via activation of a command member, from an armed position to an unarmed position, thereby enabling axial displacement of the holder. The automatic needle inserter body comprises a screw-threaded tightening ring system that enables insertion of the injection pen system into the automatic needle inserter body. The screw-threaded tightening ring system comprises an engagement surface having a proximal frustoconical inner surface which engages progressively via screw threaded action of the tightening ring against a correspondingly shaped frustoconical outer surface of a hollow deformable part, which in turn compresses a plastic ring, thereby reducing the diameter of the opening from a first wider diameter position, in which free axial movement of the pen injection system is permitted, to a second narrower diameter position, in which causes the plastic ring is pressed against, and holds, the body of the pen injection system.

As used herein, the terms “pen injection system” and “injection pen system” are used interchangeably to designate a generally handheld pen-shaped injection system, such systems being readily well known per se and commercially available for use in the treatment of many various medical indications. These systems are also often generally designed for self-injection of a drug by the user in need of treatment for the given medical indication. This is for example the case with insulin, supplied in various forms for use in the treatment of diabetes. One should note however that similarly configured injection pen systems are also available, or have been used, for the treatment of other physiological and/or pathological conditions, using a variety of injectable formulations containing active ingredients such as, for example, adrenaline, epinephrine, methotrexate, recombinant monoclonal antibodies, human growth hormone, hyaluronic acid, and the like.

Accordingly, one object of the present invention is to provide an automatic needle inserter for a pen injection system which is adapted to receive, and function with the variously shaped and dimensioned injection pen systems currently in use.

Another object of the present invention is to provide an automatic needle inserter for a pen injection system that is easier, safer and more secure to use and handle than the known solutions.

These and other objects of the invention will become readily apparent from the complete reading of the current specification.

According to any of the above objects therefore, there is therefore provided an automatic needle inserter, adapted and configured for a pen injection system as described generally above, comprising:
an elongated inserter body having a proximal extremity and a distal extremity, and a longitudinal bore extending through the elongate body from the proximal extremity to the distal extremity, the longitudinal bore having a central longitudinal axis, wherein the elongated inserter body is dimensioned and configured to receive a pen injection system introduced into said bore via the proximal extremity of the inserter body, and is further configured and dimensioned to prevent the pen injection system from exiting the longitudinal bore via the distal extremity;
the elongated inserter body further comprising:
   a rotationally-activated locking means configured to lock the pen injection system in an axial position within the longitudinal bore through rotation of at least a part of the elongated inserter body about the central longitudinal axis from a first, non-locking position to at least one or more second, locking positions.

As indicated above, the elongated inserter body is dimensioned and configured to receive a pen injection system introduced into said bore via the proximal extremity of the inserter body. Such a configuration presupposes an opening of the bore at the proximal extremity of the elongated inserter body that is sufficiently dimensioned to allow insertion of at least a distal extremity of the pen injection system into said opening and into the longitudinal bore. Whilst the general overall shape of the elongated inserter body is cylindrical, it is also possible to provide suitably dimensioned, shaped and configured widenings and/or narrowings of the bore along the length of the inserter body, dependent on the shape of the pen injection system.

Furthermore, the elongated inserter body is suitably configured and dimensioned to prevent the pen injection system from exiting the longitudinal bore via the distal extremity of the inserter body. This can be achieved in a variety of ways, for example, by providing one or more abutting shoulders which project inwardly from an inner surface of the elongated inserter body, or other forms of suitable abutment to prevent over insertion of the pen injection system along the axial length of the inserter body. A particularly advantageous solution is provided in more detail elsewhere in the present specification.

Also as indicated above, the elongated inserter body further comprises a rotationally-activated locking means configured to lock the pen injection system in an axial position within the longitudinal bore through rotation of at least a part of the elongated inserter body about the central longitudinal axis from a first, non-locking position to at least one or more second, locking positions. It is to be understood from the preceding phrase that the elongated inserted body is provided with means for rotationally locking the pen injection system in a predetermined axial position along the length of, and within, the bore of the elongated inserter body. Such rotational locking of the pen injection system against axial movement along the length of the bore is achieved via rotation of part of the elongated inserter body itself, in contrast to the solution provided for in the prior art, which uses an added on, screw-threaded ring, fitted to the outside of the proximal end of what might be considered the elongated inserter body. The functional difference between the prior art solution known from FR3079422A1 and the proposed solution of the invention is stark. Whilst both solutions involve rotation to achieve locking of the injection pen, the presently proposed solution operates in a much simpler, and easier to use manner, involving rotation of part of the elongated inserter body itself, rather than an additional element to be manipulated. To this extent, the solution proposed according to the invention functions in a manner similar to a pepper grinder, wherein a part of the elongated inserter body itself is rotated about the longitudinal axis in a first direction to operate locking of the pen, and then counter-rotated subsequent to injection in order to release the injection pen from the elongated inserter body.

Additionally, a configuration as envisaged by the present objects also provides for rotational movement from a first, non-locking position to at least one or more second, locking positions. The at least one or more, second locking positions are configured to correspond to the main diameters of injection pens currently in use. As a result, the user of the automatic needle inserter according to the invention is provided with an extremely simple to use and reliable functional solution that doesn't require the guesswork of the prior art solution, which involves the user having to determine whether or not the screw-threaded tightening ring has been tightened correctly and to the correct extent, or whether potentially the screw-thread has been over-tightened, potentially causing failure in one or more of the components. In the event of an insufficient screw-threading action, the injection pen system of the prior art risks being withdrawn from the inserter body, forcing the user to start again, or even worse, risks catastrophic failure of the inserter device, with the pen being suddenly pulled out of the inserted device and an injection needle potentially coming into accidental contact with the user in an undesired situation. Additionally, the objects as described and provided in the present specification avoid some of the other disadvantages of the prior art indicated above, among others, the requirement in the prior art solution to provide as many different tightening rings as there are diameters of injection pens. Furthermore, the prior art solution described in FR3079422A1 presents an increased risk of injection pen malfunction, as the inserter device described therein physically locks around, and potentially interferes with, some of the mechanically moving parts of the injection pen. This is in stark contrast to the objects described and presented in the present specification, which are configured to avoid physical locking contact with the moving parts of the pen injection system, instead interacting and bearing on, for example, non-deformable parts of the injection pen system, such as an outer surface of the pen's cartridge holder and/or an outer surface of the drug cartridge, thereby avoiding any risk of alteration of the selection and/or injection mechanisms of the injection pen system.

According to another object, the elongated inserter body comprises at least a first elongated outer body component, and at least a second elongated outer body component, and wherein at least one of the at least first elongated outer body component and the at least second elongated body outer component is configured to rotate during locking about the central longitudinal axis with respect to the at least other elongated outer body component. In this configuration, the elongated inserter body advantageously has two outer body components, for example, a substantially proximally located elongated outer body component extending from the proximal extremity in direction of the distal extremity of the inserter body, and a substantially distally located elongated outer body component extending from the distal extremity in direction of the proximal extremity of the inserter body, the two outer body components meeting, and being interconnected rotatably, one with respect to the other, at an appropriate point along the longitudinal axis. Advantageously, such a configuration provides for a proximal outer body component which extends towards the distal extremity of the inserter body over a distance which is greater than the corresponding distally located outer body extends in the proximal direction. In such a configuration therefore, the proximal outer body is longer than the distal outer body.

Accordingly, and in a yet further object, the elongated inserter body comprises at least a first elongated outer body component, and at least a second elongated outer body component, and wherein the at least first elongated outer body component and at the least second elongated outer body component, are configured to rotate during locking about the central longitudinal axis in opposite directions, one with respect to the other. According to such an object, the elongated inserter body functions in a manner akin to a pepper grinder, in which the user holds each outer body component in separate hands, for example, and then rotates one outer body component with respect to the other outer body component, or alternatively rotates both outer body components in respectively opposite directions, around the central longitudinal axis, from a first position to the one or more second positions.

As used herein, the reference to “outer” with regard to the elongated body signifies that these body components are radially outermost when considering the automatic inserter device as a whole, with regard to the central longitudinal axis. In turn, and as a consequence, any elements referred to as “inner”, “inside” or “inward facing” refer to parts of the device that are either contained or located within the bore formed by said outer body components, or else are directed generally inwardly into the bore formed by the outer body components.

According to another object, the rotationally-activated locking means comprises a compression ring having a nominal thickness, a nominal internal diameter defining a central bore, and wherein the compression ring is coaxially located with the longitudinal bore of the elongated inserter body. The compression ring is located coaxially within the bore of the elongated body, and where said elongated body comprises a proximal outer body component and a distal outer body component, the compression ring is advantageously completely covered and surrounded by the proximal outer body component such that an inward facing surface of said proximal outer body component comes into contact with an outward facing surface of the compression ring. In such a configuration, the outward facing surface of the compression ring is shaped and configured to engage with the inward facing surface of the proximal outer body component such that any rotation about the central longitudinal axis of the proximal outer body component is transmitted to the compression ring and causes the compression ring to rotate to the same degree, or angle of rotation, as the proximal outer body component, about the central longitudinal axis. In order to achieve this functionality, for example, the outwardly facing surface of the compression ring can be provided with one or more radially spaced apart ridges projecting from the outward facing surface of the compression ring. These ridges will engage with correspondingly appropriately shaped and configured complementary recesses provided on the inwardly facing surface of the proximal outer body component, the recesses being located at, or near the distal extremity of the proximal outer body component. Similarly, and alternatively, means for achieving the same or similar functional result can be envisaged, for example a suitable project spigot extending inwardly into the bore of the elongated outer body from an inwardly facing surface of the outer body component, said spigot engaging with a correspondingly shaped complementary groove, notch or recess, provided on the outwardly facing surface of the compression ring, whereby when the proximal outer body component is rotated, the projecting spigot engages with the corresponding complementary configured groove, notch or recess on the outwardly facing surface of the compression ring, and thereby drives rotation of the compression ring about the central longitudinal axis.

According to yet another object, the compression ring has a variable internal diameter with respect to the nominal internal diameter. By “variable internal diameter”, it is to be understood that the compression ring does not have a constant inner diameter across the bore of the compression ring, for example, due to shaping of the inward facing surface, or due to a corresponding variation of the thickness, of the compression ring extending inwardly into the bore of the compression ring.

According to yet another object, the compression ring comprises at least one portion having an increased internal diameter with respect to the nominal internal diameter.

According to yet another object, the compression ring comprises at least one portion having a reduced internal diameter with respect to the nominal diameter.

According to yet another object, the at least one portion of the compression ring having an increased internal diameter extends around the circumference of the internal diameter of the compression ring with a gradual reduction of internal diameter towards a portion of the compression ring having the reduced internal diameter. This can be understood to mean that the internal diameter is varied around the circumference of the internal diameter of the compression ring in a gradual fashion, such that said internal diameter essentially transitions from, for example, a portion of the compression ring having a diameter greater than the nominal internal diameter, and at some point along said circumference, passing through the nominal internal diameter, and then terminating in another portion of the compression ring in an internal diameter that is smaller than the nominal internal diameter. Such a transition can be provided, for example, by suitably altering the radius of curvature of the circumference of the internal diameter.

According to yet another object, the general idea expressed above of a varying internal diameter can be suitably provided, wherein the compression ring comprises at least one portion having an increased thickness with respect to a nominal thickness of the ring, extending inwardly into a central bore.

According to still another object, and in a similar and complementary manner to the preceding paragraph, the compression ring comprises at least one portion having a reduced thickness with respect to a nominal thickness of the ring, extending inwardly into a central bore.

According to yet another object, the at least one portion of the compression ring having an increased thickness extends around from an outer periphery of the compression ring inwardly into the central bore of the compression ring with a gradual reduction of thickness towards the portion of the compression ring having the reduced thickness.

According to yet another object, the rotationally-activated locking means further comprises a compressible membrane of resilient material. The compressible membrane of resilient material is provided as a contact surface for an inwardly facing surface of the compression ring. The resilient material of the compressible membrane is suitably chosen from a range of available materials, such as elastomers, for example elastomers based on thermoplastic polymers such as styrene-ethylene-butylene-styrene copolymers, commonly designated SEBS elastomers. Such elastomers show behaviors similar to rubber without having had to undergo vulcanization, and most such SEBS elastomers are generally obtained via selective hydrogenation of styrene-butadiene-styrene copolymers.

According to yet another object, the compressible membrane of resilient material is coaxially located within the bore of the compression ring and, generally ring shaped, with a bore of its own.

Further, and according to yet another object, the compressible membrane is compressed from a relaxed state to a compressed state via rotational movement of the compression ring about the central longitudinal axis from the first, non-locking position to the one or more second, locking positions.

Accordingly, the variable internal diameter of the compression ring as expressed in the various objects above, is used to provide both a variability and a control in the extent of engaging contact of the inwardly facing surface of the compression ring with an outwardly facing surface of the compressible membrane. In this manner, rotation of the compression ring, with its variable internal diameter, brings the inwardly facing surface of the compression ring, into ever increasing surface compression contact with the outwardly facing surface of the compressible membrane, causing said compressible membrane to constrict and compress about whichever object happens to be located within the bore of the compressible membrane.

According therefore to yet another object, the inwardly facing surface of the compressible membrane is brought into contact with an outside surface of a body of the pen injection system via rotational movement of the compression ring about the central longitudinal axis from the first, non-locking position to the one or more second, locking positions.

It will be understood from the above, and as envisaged in the present specification, the restriction and compression of the compressible membrane as the compression ring is rotated about the central longitudinal axis, in conjunction with the varying inner diameter of the compression ring brought to bear on the compressible membrane, causes an inner surface of the compressible membrane to be compressed and constricted onto an outer surface of the body of an injection pen system inserted into the elongated body of the automatic needle inserter. When the compression ring has been rotated from the first non-locking position, in which the pen injection system is still free to move to a certain extent about the central longitudinal axis within the bore of the elongated outer body, to the second locking position, the compression ring will have rotated with the outer elongated body, and an inner surface of the compression ring will have engaged with an outer surface of the compressible membrane, causing compression and constriction of said compressible membrane about the body of the pen injection system, thereby locking the pen injection system into position. The one or more second positions as envisaged by the objects of the present specification relate to, and are indexed with, the relative outer diameters of the major types of pen injection system currently available. The device is therefore also configurable to allow for future alternative pen diameters to be included and lockable with the various configurations of the needle inserter device as currently envisaged.

According to yet another object, the first, non-locking position and one or more second, locking positions comprise a corresponding visual or audible index, so that the user knows when any particular position has been reached. Such visual or audible index can usefully be provided, for example, by visual markings located on a corresponding relevant part of the elongated outer body, for example, at the juncture where a proximal outer body component, and a distal outer body component, meet. Such visual markings can be represented for example by notches, provided in an outward facing surface of one or more of the outer body components, and optionally supplemented by etched numbering or lettering to indicate each of the foreseen positions. If audible indexes are implemented, these can be provided by audible engagement of corresponding surfaces of the first and second elongated outer body components, said audible engagement surfaces generating an audible click, for example, as a first surface of one outer component comes into a seating or positioning contact with the second elongated outer body component. Typically, such audible index markers can comprise a projection, extending from an inward facing surface of one of the outer body components, and a corresponding groove or complementary recess on the other outer body component, such that when the projection and recess come into contact one with the other, an audible sound is generated through friction of the two surfaces one against the other, as the projection seats into the groove and/or vice-versa. For example, as envisaged by one object of the present device, a series of sequentially increasing numbers or letters can be provided on an outer surface of one of the outer body components, for example, the letters A through to E, or the numbers 0 through to 4, each number or letter representing a position corresponding to a predetermined rotation of one of the outer body components around the central longitudinal axis, and each rotational position corresponding to the required degree of compression sufficient to retain and hold the body of a pen injection system within the bore via the action of the compression ring on the compressible membrane, and corresponding to a predetermined outer diameter of the pen injection system body.

Additionally, and in order to facilitate visualization of each of the relative indexed non-locking and locking positions by the user, the outer body component not provided with such markings can usefully be provided with a magnifying surface extending therefrom over an area of the outward facing surface of the other outer body component in which said markings are provided, such as, for example, via a convex or magnifying lens. When the outer body components are moved into a corresponding unlocked, or locking position, the suitably indexed magnifying surface is located over the visual marking provided on the other outer body component, such that said marking is magnified via said magnifying surface, and thereby rendering the position marker immediately visible to the user.

According to a still further object, the compression ring is attached to, or mounted on, a slidable carriage assembly configured to translate the compression ring along the central longitudinal axis from an unarmed position to an armed position. The objective of such a slidable carriage is to provide for the automatic insertion functionality of the needle inserter. The slidable carriage assembly is therefore capable, and configured, to be movable along the central longitudinal axis from a first unarmed position, in which injection can not and does not occur, to a second, armed position, usually in a proximal direction relative to the unarmed position, in which the pen injection system is primed ready for release and subsequent movement in an opposite direction to the direction of arming, usually in a distal direction.

According to yet another object, the slidable carriage assembly comprises a bore which is coaxially aligned with the bore of the compression ring. The slidable carriage assembly, and compression ring mounted on, or attached thereto, thereby form a unitary slidable member with a common longitudinal bore.

According to yet another object, at least a part of the slidable carriage assembly is configured to co-rotate with the compression ring about the central longitudinal axis, in particular during rotational movement of the outer elongate body, from the first, non-locking position to the one or more second, locking positions. In other words, the slidable carriage assembly is shaped and dimensioned to receive the compression ring so that the latter may rotate around the central longitudinal axis during locking rotation, and corresponding unlocking, of an injection pen system inserted into the bore of the elongate body. Accordingly, the part of the slidable carriage assembly configured to co-rotate with the compression ring comprises an elongate, and substantially cylindrical body comprising a bore which is coaxially aligned with the compression ring. One way of achieving this is to provide a single rotatable moulded cylinder combining both the rotatable compression ring part and the rotatable part of the slidable carriage assembly. Alternatively, the compression ring and rotatable part of the slidable assembly can be mounted together, one with the other, for example, via suitably shaped and configured elastically deformable clips or hooks provided on either the compression ring or the rotatable part of the slidable assembly, and correspondingly shaped and configured recesses to engage with such clips in elastic deformation, such that rotation of one body, e.g. the compression ring, causes corresponding and equal rotation of the other body, i.e. the rotatable part of the slidable carriage assembly, in the same direction of rotation about the central longitudinal axis.

According to another object, the slidable carriage assembly extends respectively both in a proximal direction, and a distal direction, from the compression ring, along the central longitudinal axis. The proximally extending part of the slidable carriage assembly is the rotatable part described above in relation to the compression ring. The slidable carriage assembly can thus be considered as an assembly of a proximal part, and a distal part, with the compression ring located in-between the proximal part and the distal part. In such a configuration, the first, proximal part has a cylindrical shaped body, with a central longitudinal bore as described above, and co-rotates with the compression ring. The proximal part also comprises a distal end comprising, for example, an inwardly facing annular groove. The annular groove is suitably configured and dimensioned to receive two or more radially spaced apart projecting arcuate walls extending from the second, distal part of the slidable carriage assembly in a proximal direction, and terminating in a radially outwardly projecting spar or hook portion. During assembly of the device, the projecting arcuate walls of the second, distal part of the carriage body are inserted through the bore of the compression ring, and into the coaxially aligned bore of the first, proximal and rotatable part of the slidable carriage assembly, whereby the outwardly projecting spar or hooks provided at the proximal ends of the arcuate walls engage in the annular groove of the first, proximal part of the slidable carriage assembly. The arcuate walls and outwardly projecting hook portions at the proximal ends thereof engage with the annular groove in non-rotationally blocking engagement, i.e. the projecting hook portions are free to move along the groove in a clockwise or counter-clockwise direction when the compression ring and first, proximal part of the slidable carriage assembly are rotated about the central longitudinal axis in a corresponding clockwise or counter-clockwise direction.

The second, distal part of the slidable carriage assembly is additionally provided with a radially outwardly projecting ridge, which forms a proximally facing distal abutment surface for a distal extremity of the compression ring. As the first, proximal and rotatable part, and the second, distal part of the carriage body are maintained in relative axial separation one with regard to the other, the compression ring is trapped against independent axial movement along the longitudinal axis. This is more so the case when the compression ring and proximal rotatable part of the slidable carriage assembly are constituted from a single moulder cylinder. The projecting arcuate walls of the second, distal part of the carriage body advantageously provide a convex curved, outwardly facing surface against which the compression ring can rotate during positional locking and unlocking of the injection pen body. Another advantageous feature of the radially spaced apart projecting arcuate walls extending from the second, distal part of the slidable carriage assembly is that the spaces provided between said extending arcuate walls are dimensioned to receive, and retain by sidewards compression, a respective corresponding radially outwardly extending projection of material of the compressible resilient membrane. Such a radially outwardly extending projection of the material constituting the compressible resilient membrane corresponds to the outwardly facing contact surface which comes into contact with the compression ring during rotation of the compression ring, and which duly transmits the compression force applied progressively by the compression ring to cause the membrane to be compressed and reduce the diameter of the bore of the compressible membrane onto the body of the pen injection system. The compressible membrane can suitably be provided with two or more, for example, four or six, or eight, outwardly extending, radially spaced apart, projections of compressible material. In one particularly advantageous embodiment, the compressible material can be moulded as a cylinder with a corresponding longitudinal bore which is coaxially aligned with the central bore, onto an inwardly facing surface of the arcuate walls.

As will be understood from the above, if the slidable carriage assembly moves in an axial direction, irrespective of whether this is in a proximal or distal direction, the compression ring is forced to move axially and in translation with the carriage assembly to the same extent.

According to yet another object, the slidable carriage assembly and compression ring each comprise surface engagement means configured to engage in sliding engagement with the first elongated outer body component, to enable translation of the compression ring, along with the slidable carriage assembly, from the unarmed position to the armed position, without rotation of either the compression ring or the slidable carriage assembly within the bore of the first elongated outer body component. Such surface engagement means, which are generally complementary to each other, can be suitably provided in a number of ways.

For example, and according to another object, the surface engagement means of the slidable carriage assembly comprise at least one projecting contact member extending radially outwardly from the slidable carriage assembly.

According to another object, the at least one projecting contact member extending radially outwardly from the slidable carriage assembly engages in axial sliding contact with at least one corresponding runnel provided on the first elongated outer body component. The at least one corresponding runnel as foreseen herein extends along, and in parallel to the longitudinal axis, at least part of an inwardly facing surface of the first outer body component. The runnel, which receives the projecting contact member of the slidable carriage assembly, surrounds the projecting contact member and prevents rotation of the carriage assembly about the central longitudinal axis, as the carriage assembly translates from the unarmed position to the armed position.

According to another object, the at least one projecting contact member extending radially outwardly from the slidable carriage assembly also extends proximally beyond a proximal extremity of the proximal part of the slidable carriage assembly. From this, it is to be understood that the at least one projecting radially outwardly projecting contact member can advantageously be represented as a series of radially spaced and outwardly projecting legs, positioned for example, on an outwardly facing surface of the first, proximal part of the slidable carriage assembly, and furthermore extending along said first, proximal part of the carriage assembly in parallel to the central longitudinal axis beyond the proximal end of the first, proximal part of the carriage assembly. The length of the projecting legs extending beyond the proximal extremity of the carriage body is configured, for example, to provide an appropriate abutting distance of the proximal end of the carriage body with the proximal end of the outer elongate body, or alternatively, to provide a proximal engagement surface with a biasing element such as a compression spring located within the bore at the proximal end of the outer body, and furthermore to limit the distance of travel of the carriage body in the proximal direction.

Similarly to the projecting contact members provided on the slidable carriage assembly, and according to yet another object, the compression ring comprises at least one projecting contact member extending radially outwardly from the compression ring. As has been explained above when describing the compression ring in relation to the proximal outer body component, the outwardly facing surface of the compression ring can be provided with one or more radially spaced apart ridges projecting from the outward facing surface of the compression ring. These ridges will engage with correspondingly appropriately shaped and configured complementary, radially spaced apart recesses, or runnels, provided on the inwardly facing surface of the proximal outer body component, the recesses or runnels extending along the inwardly facing surface of the proximal outer body component, in parallel to the longitudinal central axis, from the distal extremity of the proximal outer body component to the proximal extremity thereof.

According to still a yet further object, the at least one projecting contact member extending radially outwardly from the compression ring engages in axial sliding contact with at least one corresponding runnel provided on the first elongated outer body component.

According to another object, the slidable carriage assembly comprises at least one slider arm extending distally from a distal part of the carriage assembly, radially spaced apart from, and in parallel to, the central longitudinal axis. Whilst it is possible to configure an automatic needle inserter according to the present specification having only one such slider arm, it has been found useful, in order to stabilize translational travel of the slidable carriage assembly along the longitudinal axis, to provide two or more such slider arms, extending from the distal extremity of the carriage assembly, such arms generally being positioned radially equidistant around the central longitudinal axis. The slider arms advantageously extend distally from the radially projecting ridge provided on the second, distal part of the slidable carriage assembly, and are located radially spaced apart, and substantially opposite one another, around the central longitudinal axis. Additionally, said slider arms are also advantageously located in angular opposition to the radially spaced apart, arcuate projecting walls extending from the second, distal part of the carriage body in a proximal direction. In other words, if considering the points of a clock face about which the slider arms and the arcuate walls would be distributed, the slider arms would be located at 12 o'clock and 6 o'clock respectively, extending forward from the clock face, and the arcuate projecting walls would be located at 3 o'clock and 9 o'clock extending rearwards from, and respectively about, the clock face. Where more than two pairs of arcuate walls are provided in the second, distal part of the slidable carriage assembly, the slider arms which extend in a distal direction can be suitably located at angular, or clock positions, between any of these pair of proximally extending arcuate walls.

According to another object, the at least one slider arm engages in sliding axial contact with at least one corresponding runnel provided on the second elongated outer body component. The corresponding runnel or runnels provided on the second elongated outer body component extend from a proximal extremity of the second elongated outer body component towards a distal extremity of said second outer body component, and are suitably provided on an inward facing surface of said second outer body component. The slider arm or arms, engage slidably within the correspondingly positioned runnel, or runnels, thereby permitting sliding or translational movement of the carriage assembly, both in a proximal and distal direction, whilst simultaneously preventing said second outer body component from rotating around the central longitudinal axis. The slidable, or translational movement, of the carriage assembly along the longitudinal axis in the distal direction is limited by the length of the slider arms and the corresponding runnels, a distal extremity of the slider arm forming an abutting stop with a distal extremity of a corresponding runnel provided on the second outer body component.

According to another object, the at least one slider arm has a length sufficient to extend distally into, and maintain sliding engagement contact with, the corresponding runnel provided on the second elongated outer body component, when the slidable carriage assembly is in the armed position.

According to yet another object, the slidable carriage assembly further comprises a releasable trigger means having a trigger member, configured to retain the slidable carriage assembly in the armed position until the trigger member is released. The releasable trigger member is provided to enable the user to cause the pen injection system to move in a distal direction from the armed position through to an injection position in which the needle of the pen injection system penetrates an injection surface such as the skin, to the correct and/or desired depth of penetration. Usually, the impetus for moving the pen injection system from the armed position to the unarmed position via an injection operation, is provided by a biasing element, such as, for example, a compressed spring. As envisaged in the present specification, the slidable carriage assembly is moved into the armed position, for example, by the user of the inserter device pulling or exerting traction, on the pen injection system as held in the slidable carriage assembly by the compression ring and compressible membrane, after the former has been rotated into the locked position, said traction being exerted in a proximal direction. In essence, the user of the automatic needle inserter pulls the pen body backwards, in a proximal direction, against the biasing element, compressing the latter against the proximal end of the inserter body, until the trigger member of the releasable trigger is latched, and the armed position set.

According therefore to yet another object, the trigger member comprises an elastically deformable arm that is movable out of a first plane of longitudinal axial alignment in the unarmed position into a second plane of longitudinal axial alignment in the armed position. The elastically deformable arm can advantageously extend from the proximal part of the slidable carriage assembly in a proximal direction, for example, either extending directly along the longitudinal axis from said proximal part of the slidable carriage assembly, or alternatively, extending floating from an extremity of a projecting spur which extends orthogonally from an outer surface of the proximal part of the slidable carriage assembly, across the central longitudinal axis, and which aligns the elastically deformable arm to said central longitudinal axis. The elastically deformable arm lies along a first longitudinal plane, which is in parallel to the central longitudinal axis in the unarmed state, and before deformation. As the slidablye carriage assembly is moved in a proximal direction, the elastically deformable arm meets an abutment, such as a sloping shoulder, provided on an inwardly facing surface of, and adjacent to, the proximal extremity of the outer elongate body. The abutment, which lies in the same longitudinal plane as the elastically deformable arm, causes the arm to deform and be moved out of the first longitudinal plane into a second longitudinal plane which is radially separate from the first longitudinal plane. As the arm is moved proximally due to the proximal movement of the slidable carriage assembly, so the arm comes into deflecting or deforming contact with the sloping shoulder of the of the first outer body component. The arm is deformed so far out of the first plane into the second plane until a proximal extremity of the arm, for example, a hook-shaped portion, moves past the shoulder and a corresponding inversely-shaped hook portion provided on the inside surface of the proximal outer body. The hook of the arm then engages with the corresponding and inversely shaped hook of the shoulder, causing the trigger member to lock the slidable carriage assembly in the armed position in said second longitudinal plane, in which the hook of the arm is subjected to a continuing mechanical deformation constraint.

According to yet another object, the first elongated outer body component comprises a release button configured to move the elastically deformable arm out of the second plane of longitudinal axial alignment into the first plane of longitudinal axial alignment, and thereby release the trigger member from the armed position.

As mentioned above, one way of achieving such functionality is for a latch or hook to be provided on the trigger member, which engages with the release button provided on the elongate outer body, and maintains the arm of the trigger member under mechanical constraint in the second longitudinal plane until such time as the release button is activated, for example, by a user pressing the release button. As the elastically deformable arm in the armed position was under an elastic mechanical constraint forced by the move from the first plane to the second plane, when the release button is activated, the elastically deformable arm is freed from such elastic constraint and assumes its normal position in the first longitudinal plane once more. In so doing, the carriage assembly is now free to be moved, by the energy stored by the compression spring, in a distal direction, to accelerate the carriage assembly, and correspondingly held injection pen to move the injection needle into a correctly configured depth of penetration.

According to another object, the automatic needle inserter comprises a selectively actionable pen distal extremity abutment means configured to abut a distal extremity of the injection pen system, upon insertion of the pen injection system into the longitudinal bore of the elongated inserter body, along the central longitudinal axis of the longitudinal bore. The term “selectively actionable” as used herein is to be understood as meaning that the pen distal extremity abutment means functions to abut the distal extremity of the pen injection system along the central longitudinal axis when introduced into the elongated inserter body, in a selectively active manner, i.e through a deliberate user action or interaction with the abutment means with regard to the elongated inserter body. In other words, the abutment means is not an element or object that is permanently active or forming part of the inserter body that constantly exerts an abutting action on any introduced injection pen system, rather it can be activated or deactivated, as and when required. In general, the pen distal extremity abutment means is activated before introduction of the pen injection system into the elongated body of the inserter, and deactivated after the injection pen body has been locked into the second, locking position via rotation of a part of the elongated outer body from the non-locking position to the locking position as described elsewhere in the present specification.

According therefore to yet another object, the rotationally activated locking of the pen injection system occurs only after abutment of the pen injection system via selective actioning of the pen distal extremity abutment means.

According to yet another object, the selectively actionable pen distal extremity abutment means is movable from a first, non-abutting position, to a second, abutting position. Such movement can be imparted, for example, by the user through a finger or thumb movement, or a combination of finger and thumb movement. Similarly, the pen distal extremity abutment means is selectively actionable in a reverse, or opposite, direction, from the second, abutting position, to the first, non-abutting position. In this way, the pen distal extremity abutment means are configured to enable repositioning of the abutment means into a non-abutting position once the pen injection system has been locked into position.

According to yet another object, the selectively actionable pen distal extremity abutment means is located adjacent, or in proximity, to the distal extremity of the elongated inserter body, in order for abutment to occur as closely as possible to a distal extremity of the pen injection system, and more particularly, as closely as possible to a needle mount shoulder generally located at the distal extremity of such injection pens, onto which an injection needle is mounted, for example, by screw threading engagement, with said needle mount. In this way, the pen distal extremity abutment means when activated interacts with a part of the injection pen system that will define the depth of penetration of the needle into the injection site, for example, the skin of a patient, when the injection pen is released from the armed position, and is moved towards the unarmed position, under the impulsion of the biasing element, such as the compression spring. This depth of penetration can therefore be controlled and predetermined in advance, as the length of the injection needle mounted on such needle mounts is generally standardized, and the abutment of the needle mount of the pen injection system before arming of the inserter device will be positioned at a known predetermined axial distance from the site of future injection.

According to yet another object, the selectively actionable pen distal extremity abutment means comprises an articulated arm member, configured to rotate about an axis of rotation in parallel alignment to the central longitudinal axis, and wherein the articulated arm member is rotatable about said parallel axis of rotation from the first, non-abutting position, to the second, abutting position. In accordance with such an object, the articulated arm member can be suitably mounted on an axis of rotation that lies parallel to the central longitudinal axis, and is rotatable about said parallel mounting axis to be movable, by rotation, for example by finger or thumb interaction with the arm, to move the arm from a non-abutting engagement, for example, essentially lying flush against the outer elongated inserter body, to an abutting position in which the arm projects into the bore of the outer elongated inserter body.

According therefore to yet another object, the articulated arm member lies flush with the elongated inserter body in the first, non-abutting position, and extends into the longitudinal bore in the second, abutting position.

Such a selectively actionable, and rotatable arm configuration can be suitably provided by a lever arm. For example, the lever arm can comprise a first prehensile end, which in the non-abutting position lies essentially flush with an outwardly facing surface of the elongated inserter body, and a second, abutting end, which lies essentially flush with an inwardly facing surface of the outer elongated inserter body in said non-abutting position. A lifting action, for example, exerted by the user on the first prehensile end of the lever arm, causes the lever arm to rotate about the longitudinal rotational axis on which the arm is mounted, and move the second end of the lever arm from the essentially flush position on the inwardly facing surface of the elongated outer body of the inserter, into the bore said elongated inserter body. In order to maintain the lever arm in either the non-abutting, or abutting positions, the lever arm can further comprise at least one positioning nodule, for example extending orthogonally outwards from the plane in which the lever arm lies on the inserter body, and appropriately positioned on the lever arm, so that the at least one positioning nodule is lodged within at least one corresponding positioning recess provided within the elongated inserter body, and thereby determines the angle of of movement of the lever arm about the rotational axis, which in turn determines the extent to which the second end of the lever arm is moved into the bore of the elongated inserter body.

These and other objects of the invention will become apparent and described in more detail in the following description relating to the figures and an example monitoring module.

### BRIEF DESCRIPTION OF THE FIGURES

The invention will now be described in more detail with regard to the accompanying figures, provided for the purpose of illustration and exemplification, in which:

FIG. 1 is a schematic exploded perspective representation of an automatic needle insertion device according the present invention;

FIGS. 2A and 2B, are respectively, a schematic perspective representation and a schematic cross-sectional representation of the automatic needle insertion device according to FIG. 1, in an unarmed position;

FIGS. 3A and 3B are respectively, a schematic perspective representation and a schematic cross-sectional representation of the automatic needle insertion device according to FIG. 1, in an armed position, ready for injection;

FIGS. 4A and 4B are respectively, a schematic, exploded perspective representation, and a schematic, perspective assembled representation of a detail of the automatic needle insertion device according to FIG. 1.

FIGS. 5A and 5B are respectively, schematic cross-sectional representations of another detail of the automatic needle insertion device according to FIG. 1.

### DETAILED DESCRIPTION OF AN EXAMPLE

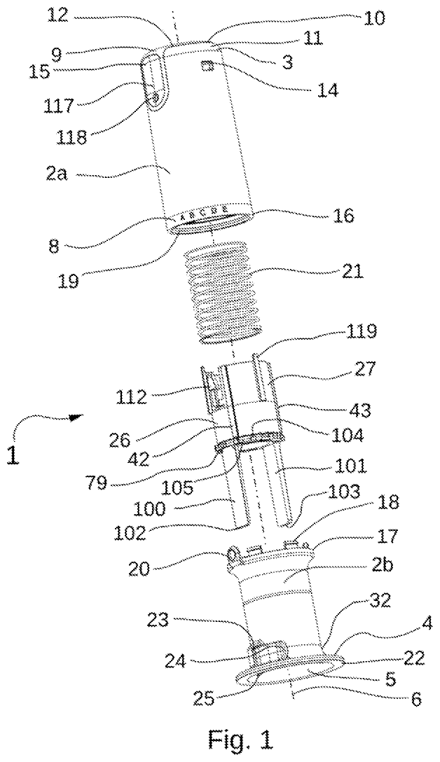

Turning now to the figures, FIG. 1 illustrates a schematic exploded perspective representation of an automatic needle inserter device (1) according to the invention. The automatic needle inserter (1) comprises an elongated inserter body (2a, 2b) having a proximal extremity (3) and a distal extremity (4), and a longitudinal bore (5) extending through the elongate body (2a, 2b) from the proximal extremity (3) to the distal extremity (4), the longitudinal bore (5) having a central longitudinal axis (6), wherein the elongated inserter body (2a, 2b) is dimensioned and configured to receive a pen injection system (7) introduced into said bore (5) via the proximal extremity (3) of the inserter body (2a, 2b). The elongated inserter body (2a, 2b) further comprises a rotationally-activated locking means, which will be described in more detail herein, configured to lock the pen injection system (7) in an axial position within the longitudinal bore (5) through rotation of at least a part of the elongated inserter body (2a, 2b) about the central longitudinal axis (6) from a first, non-locking position (8), referenced by a dot (•) to at least one or more second, locking positions, as referenced by a series of visual markers (A, B, C, D, E). Each visual marker corresponds to one of the second locking positions and, more particularly, represents the rotational locking position for an injection pen system of a predetermined outer diameter of a body of the injection pen. In essence, each visual marker corresponds to an angle of rotation about the central longitudinal axis, set to, for example, an increment of 15° per rotational locking position. The proximal extremity (4) of the elongated inserter body is closed by a closure cap (9) having an annular shoulder (10) extending from a peripheral annular wall (11) inwards into the bore (5), and defining an opening (12) through which the pen injection system (7) is introduced into the inserter body (2a, 2b). The peripheral annular wall (11) extends in a distal direction from the annular shoulder (10) and is provided with at least one elastic engagement clip, latch or hook (13), to engage in elastic clip engagement with a corresponding recess (14) or lip provided on the elongate body (2a), the peripheral annular wall (11) being dimensioned so that it can be inserted into the bore (5). The cap (9) also comprises a release button (15), enabling a user to activate release of the inserter device to effect introduction of the needle of the pen injector into a site of injection, for example, on the skin of a patient, the functioning of which will be described in more detail hereafter.

As is apparent from FIG. 1, the elongated inserter body (2a, 2b) comprises two components: a first proximal, outer body component (2b), and a second, distal outer body component (2b). The first outer body component (2a) and second outer body component (2b) are interlocked together at a respective distal (16), and proximal (17) end, for example, as illustrated, projecting elastically engaging teeth (18) or hooks extending from the proximal end of the second outer body component (2b) and a corresponding annular lip or groove (19) provided at the distal end (16) of the first outer body component (2a). The elastically engaging teeth (18) engage with the annular groove (19) to allow rotation of either, or both, of the first and second outer body components (2a, 2b), around the central longitudinal axis (6), one with respect to the other. The second body component (2b) is further provided with a rotational position identifying aid (20), such as a magnifying lens, to assist in determination of the position in which the first and second body components (2a, 2b) have been rotated, one with respect to the other.

Also illustrated in FIG. 1 is a compression spring (21) which is introduced into the bore (5) and which is positioned and seated at the proximal end (3) of the elongate outer body (2a), at least partly within the cap (9), and which serves to provide an energy store to release energy after being compressed, when the injection pen (7) is released from the armed position within the inserter to introduce an injection needle into an injection site and return the automatic needle inserter device (1) and correspondingly held injection pen (7) to the unarmed position, as will be described in more detail hereafter.

The distal end (4) of the second outer body component (2b) is furthermore shaped to resemble the exit end of a trumpet and comprises to that effect an outwardly projecting annular shoulder or skirt (22), the skirt extending radially outward from an outer surface of the second outer body component (2b). The skirt thereby forms a suitable stable positioning surface for positioning of the automatic needle inserter on an injection site of a patient or user. Additionally, a pen distal extremity abutment means in the form of a lever arm (23) is located at the distal extremity (4) of the second outer body component (2b), the lever arm comprising a first prehensile end (24) and a second, abutting end (25), details of which will be provided hereafter.

Finally, FIG. 1 also illustrates further details of the locking system, and arming system of the automatic needle inserter, as represented by a compression ring (26), and a slidable carriage assembly (27), the details of which will be provided when referring to the other figures.

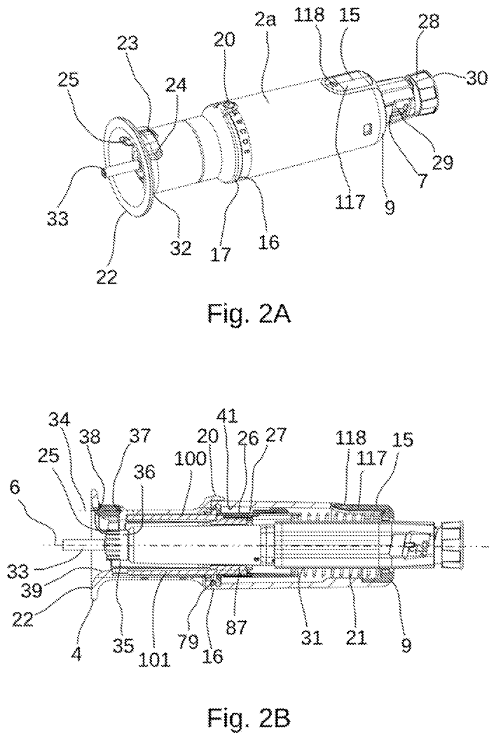

FIGS. 2A and 2B illustrate, respectively, two similar, but slightly different views of the automatic needle inserter device according to FIG. 1. FIG. 2A is a schematic perspective view in which an injection pen system (7) can be seen having been introduced via the opening (12) of the closure cap (9) into the bore of the elongated inserter body (2a, 2b), and is in an unarmed position, i.e. not positioned to effect an automatic insertion of the needle of the injection pen system (7). The injection pen system (7) as illustrated here, and like many other such pens currently available in commerce, comprises a dose setting wheel (28), a dose visualization window (29) and an injection activation button (30), which are all visible and accessible to the user and which project outside of the elongated inserter body (2a, 2b) beyond the proximal extremity (3) of the proximal body component (2a) and through the opening (12) in the cap (9). The release button (15) is also visible at the proximal end (4) of the elongated inserter body (2a, 2b). The proximal (2a) and distal (2b) outer body components are connected together at their respective and corresponding distal (17) and proximal (17) ends to form an elongated cylinder surrounding and receiving most of the body (31) of the injection pen (7), apart from a proximally projecting portion of the body which extends beyond the opening (12) of the closure cap (9). This projecting portion of the injection pen (7) is held by the user and drawn or pulled in a proximal direction towards the user, whilst the user holds the proximal elongate outer body component (2a), when arming the automatic inserter device (1). FIG. 2A also shows a pen distal extremity abutment mechanism for the injection pen comprising a lever arm (23), having a first prehensile end (24), in a first, non-abutting position in which the prehensile end (24) of the lever arm (23) lays essentially flush with an outer surface (32) of the distal outer body component (2b). A needle (not shown) covered by a needle guard (33) can be seen protruding from the distal extremity (4) of the distal outer body component (2b) of the elongate body (2a, 2b).

FIG. 2B shows a schematic cross-sectional view of automatic needle inserter device as illustrated in FIGS. 1 and 2A. One noticeable difference between FIG. 2A and FIG. 2B is that the lever arm (23) of the pen distal extremity abutment means has been rotated by manipulating the prehensile end (24), and causing the lever arm to rotate about a rotation axis (34) which lies parallel to the central longitudinal axis (6), so that the abutting end (25) of the lever arm has been brought into contact and pushes against an outside surface (35) of a needle mount (36) located at a distal extremity of the pen injection system (7). The distal outer body component (2b) of the elongate body (2a, 2b) is provided with appropriately configured and dimensioned opposing recesses (37, 38) to serve as fulcrum points about which the lever arm (23) can rotate when activated. The lever arm (23) is suitably provided with corresponding and complementary projections (not shown) extending orthogonally outwardly from the body of the lever arm, defining with the recesses (37, 38) the parallel axis (34) of rotation. From the above, it will be understood that activation of the lever arm (23), through a rotation applied by the user lifting the prehensile end (25) about the parallel axis of rotation (34), causes the abutting end (25) of the lever arm (23) to move from a non-abutting position, in which the abutting end (25) lays essentially flush with an inner surface (39) of the distal outer body component (2b), to an abutting position, in which the abutting end (25) is brought into contact with, and pushes against, the outside surface (35) of the needle mount (36). In this way, the injection pen system (7) is abutted along the central longitudinal axis (6) before it is rotationally locked into an axial position along the central axis (6), as will be described in more detail hereafter.

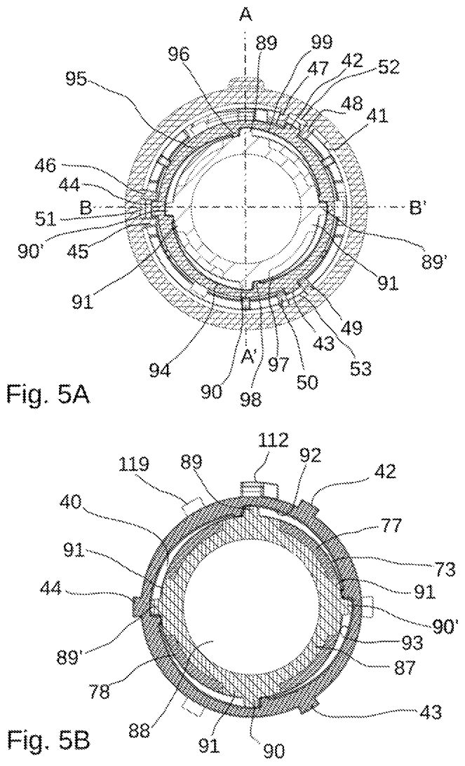

FIG. 2B, and as also illustrated in further detail in FIGS. 5A and 5B, represents a schematic cross section of the rotationally activated locking means of the automatic needle inserter, one component of which is a compression ring (26) having a nominal thickness and a nominal internal diameter defining a central bore (40). As can be seen from FIG. 2B, the compression ring (26) is coaxially located with the longitudinal bore (6) of the elongated inserter body (2a, 2b) and, in the unarmed position of the needle inserter device (1), is located in a distal region of the proximal outer body component (2a), close to the distal extremity (16) of the proximal outer body component (2a). In the unarmed position, the compression ring (26) is free to rotate about the central longitudinal axis (6).

As illustrated in greater detail in FIGS. 5A and 5B, when the proximal outer body component (2a) is rotated about the central longitudinal to initiate rotational locking from the first, unlocked position to the one or more second, locked positions, which locked positions correspond, and are suitably indexed, to the outer diameters of various injection pens currently available, and which are intended to be introduced into the automatic needle inserter device, an inwardly facing surface (41) of the proximal outer body component (2a) interacts and engages with one or more radially, and outwardly, facing projections, indicated in FIGS. 5A and 5B (42, 43, 44). The inwardly facing surface (41) of the proximal outer body component (2a) is provided with one or more spaced apart, radially inwardly facing ridges (45, 46, 47, 48, 49, 50), extending axially in parallel at least partly along the central longitudinal axis (6) from a position located proximally of the proximal extremity (16) of the proximal outer body component (2a) and defining between each pair of ridges, a corresponding groove or runnel (51, 52, 53). The bore (40) of the compression ring has an internal diameter sufficiently dimensioned to be able to receive at least a part of the slidable carriage assembly (27) within the bore (40), and permitting rotation of the compression ring about the central longitudinal axis (6) within the bore of the proximal outer body component (2a) in the unarmed position.

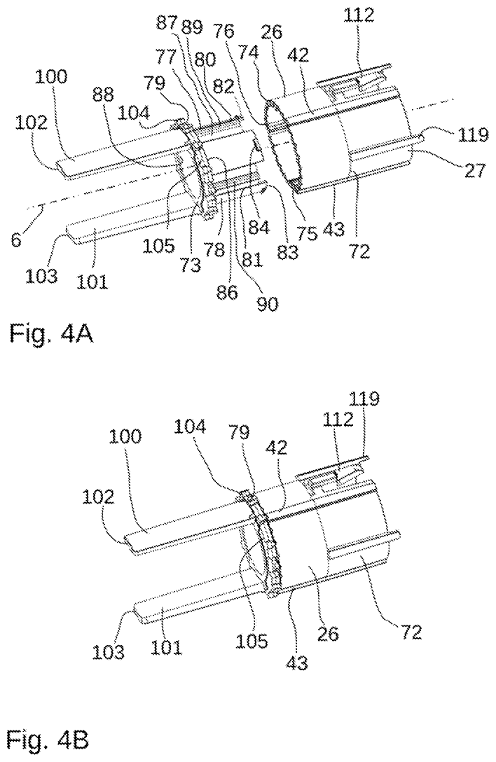

In the embodiment shown in FIGS. 4A and 4B, the slidable carriage assembly (27) has a carriage body (72, 73) comprising a proximal part (72) and a distal part (73). The proximal part (72) and distal part (73) are assembled coaxially to, and along, the central longitudinal axis to define an arcuate area around which the compression ring (26) is located. As can be seen from FIG. 4A, the proximal part (72) of the carriage assembly is provided with a distal extremity (74) comprising an inwardly projecting annular shoulder or skirt (75), the shoulder (75) defining a distally facing surface (76). The distal part (73) of the carriage body is advantageously provided with a pair of diametrically opposing, radially spaced apart, arcuate walls (77, 78) extending in the proximal direction from a distally located, radially outwardly projecting, ridge (79). The lengthwise edges of each of the opposing arcuate walls (77, 78) define at least one pair, and preferably at least two pairs, diametrically opposing spaces (80, 81) there-between, and the proximal ends (82, 83) of the arcuate walls (77, 78) are received in an annular groove (not shown) provided in the annular shoulder (75). The proximal ends (82, 83) of the arcuate walls can be suitably provided with radially outwardly extending hooks (84), and are received in the corresponding annular groove of the shoulder (75), for example. In this way, a proximal facing surface (86) of the ridge (79) and the distal facing surface (76) of the skirt (75), along with the arcuate walls (77, 78) define an area for receiving and locating the compression ring (26). In a particularly advantageous aspect, the proximal part (72) and the compression ring (26) are unitary, coaxially aligned, and configured to rotate together about the central longitudinal axis (6) during rotational locking.

The rotational locking means also comprises a compressible membrane (87) of resilient material, such as an elastomer, for example, as a SEBS elastomer, as described elsewhere in the present specification. The compressible membrane is advantageously formed in the shape of a ring, and has a bore (88). The compressible membrane ring (87) is located within the bore of the slidable carriage assembly (27) and, more particularly, is seated in non-rotational engagement in the bore of the proximal part (73) of the carriage body, in between the arcuate walls (77, 78). The compressible membrane ring (87) further comprises at least one, and preferably one, two, three, or even four pairs, of diametrically opposing radially outwardly extending projections (89, 90) of compressible material, which even more advantageously can be made of a SEBS elastomer having a lower compressibility, or a higher Shore hardness, than the remainder of the compressible membrane ring. The diametrically opposing radially outwardly extending projections (89, 90 and 89′, 90′) and of compressible material can be formed, and extend, for example, from a shoulder (91) of reduced thickness compared to the projections, extending at least part way around the outer circumference of the ring (87), the shoulders (91) advantageously lying flush with the outer surface of the arcuate walls (77, 78) to form a continuous arcuate surface. The shoulder (91) serves as a contact surface that engages with a peripheral edge of the arcuate walls (77, 78), thereby locating the radially outwardly extending projections (89, 90) into the spaces (80, 81) formed by the edges of the arcuate walls, such that the compressible membrane ring (87) can not rotate relative to the arcuate walls (77, 78).

As will be understood from the above, the compression ring (26), during rotation from the non-locking position to the one or more second, locking positions, is therefore brought into contact with the exposed radially outwardly extending projections (89, 90; 89′, 90′) of the compressible membrane ring (87). The inner diameter of the compression ring (26) is variable around the inner circumference defining said inner diameter. In the exemplified and illustrated embodiments, this variation is represented by a gradual thickening of the ring of the compression ring about the central axis (6), from a portion (FIG. 5A, 94) of greater inner diameter than the nominal inner diameter to a portion of lower, or reduced diameter than the nominal inner diameter, said portion (95) having an increased thickness, and advantageously an inwardly facing projection (96). Similarly, a diametrically opposed gradual thinning of the compression ring is provided from a portion (97) of the compression ring having reduced inner diameter, i.e. increased thickness, compared to the nominal inner diameter, and having a corresponding thickened inwardly facing projection (98), to a portion (99) of the compression ring having a greater inner diameter, i.e.; reduced thickness, compared to the nominal inner diameter. Due to gradual variation in inner diameter, the compression ring (26) applies gradual compression to the radially outwardly facing projections (89, of the compressible membrane (87) as the compression ring (26) is rotated about the central axis (6) in a first direction, by engagement of an inner circumferential surface of the compression ring (26) with the radially inwardly projecting ridges (89, 90) of the proximal outer body component (2a). The thus applied radially directed compression causes the compressible membrane (87) to be deformed radially inwardly and move an inwardly facing surface of the compressible membrane to come into elastic frictional engaging contact with an outside surface of the body of the pen injection system (7). Over-rotation, and hence over-compression of the compressible membrane (87), can be prevented via the inwardly facing projections (96, 98), which are provided with corresponding rotational abutment surfaces, which come into contact with the outwardly facing projections (89, of the compressible membrane, et thereby limit the angle of rotation of the compression ring (26) about the central axis (6). It will be understood from the above that rotation of the compression ring in the opposite direction causes relaxation of the compression exerted on the compressible membrane (87), thereby removing elastic frictional engaging contact of the compressible membrane with the body of the pen injection system (7), allowing the latter to be removed from the automatic needle inserter device, for example for storage or exchange of the pen for future injections.

The slidable carriage assembly (27) also comprises two slider arms (100, 101) extending distally and radially equally spaced apart, distally from and attached to the distal part (73) of the slidable carriage assembly, at an outwardly projecting ridge (79) also provided on the distal part of the slidable carriage assembly. The slider arms (100, 101) extend in parallel to the central longitudinal axis (6), and are located radially around said axis (6). The slider arms (100, 101) terminate in a corresponding pair of abutment edges (102, 103). The two slider arms (100, 101) help to stabilize translational travel of the slidable carriage assembly along the central longitudinal axis (6), and prevent rotation of the slider assembly around the central longitudinal axis (6), after rotational locking has been carried out, and when moving the slidable assembly from the unarmed position to the armed position. Additionally, the slider arms (100, 101) are also advantageously located in angular opposition to the radially spaced apart, arcuate projecting walls (77, 78) extending from the distal part (73) of the carriage body in the proximal direction. In other words, if considering the points of a clock face about which the slider arms and the arcuate walls would be distributed, with the central axis (6) as the centre of the clock face, the slider arms (100, 101) would be located at 12 o'clock and 6 o'clock respectively, extending forward from the clock face, and the arcuate projecting walls (77, 78) would be located at 3 o'clock and 9 o'clock extending rearwards from, and respectively about, the clock face. In such an imagined projection, the arcuate walls (77, 78) would extend around the clock face to form an arc shaped surface from about 1 o'clock to about 5 o'clock, and from about 7 o'clock to about 11 o'clock, respectively, with the gaps in between representing the spaces (80, 81) configured to receive the radially outwardly extending projections (89, 90) of the compressible membrane (87). The ridge (79), forming the distal extremity of the distal part (73) of the carriage body, comprises a series of ridges (104) and grooves (105), the ridges projecting radially outwardly. The ridges (104) and grooves (105) of distal extremity ridge (79) engage in sliding contact with correspondingly shaped grooves or runnels and ridges provided on the inward facing surface (41) of the outer body component (2a), to facilitate translation of the slidable carriage assembly within the bore of proximal outer body component (2a), but without impeding rotation and rotational engagement of the proximal outer body component (2a) with the compression ring (26) during rotational locking and unlocking. The differential distribution and extension of the grooves and ridges of the proximal outer body component (2a) will be chosen to enable rotation of the compression ring (26) about the central axis, in order to effect rotational locking and unlocking, whilst also providing for translation of the slidable carriage assembly (27) along the central axis (6), along with the compression ring (26), in order to effect arming and needle insertion and/or disarming.

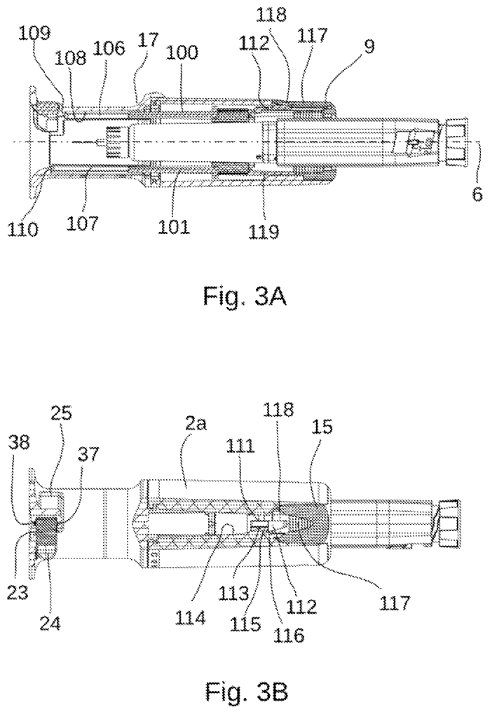

As illustrated in FIG. 3A, which shows the pen injection system (7), and various components of the slidable carriage assembly, in the armed position, the slider arms (100, 101) engage in sliding axial contact with corresponding runnels (106, 107) provided on the distal elongated outer body component (2b). The runnels (106, 107) extend from the proximal extremity (17) of the distal elongated outer body component (2b) towards the distal extremity (4) of said distal outer body component, and are suitably provided on an inward facing surface (108) of said distal outer body component. The slider arms (100, 101), slidingly engage within the correspondingly positioned runnels (106, 107), thereby permitting translational movement of the carriage assembly (27), both in a proximal and distal direction. Given that the distal part (73) of the carriage assembly is translationally coupled to the proximal part (72) of the carriage assembly via the proximal ends of the arcuate walls (77, 78), which engage in the annular groove (84) via their corresponding outwardly projecting hooks or spurs, the slidingly engaged arms prevent the distal outer body component (2b) from rotating around the central longitudinal axis (6) with respect to the proximal outer body component (2a) during arming of the inserter device, and release of the slidable carriage assembly back to the unarmed position. The sliding, or translational movement, of the carriage assembly (27) along the longitudinal axis in the distal direction is furthermore limited by the length of the slider arms (100, 101) and the corresponding runnels (106, 107), each slider arm having a distal extremity (102, 103) which forms an abutting stop with a corresponding distal extremity (109, 110) of a corresponding runnel (106, 107) provided on the distal outer body component (2b). It will thus be understood from the above that the slider arms (100, 101) have a length which is sufficient to extend distally into, and maintain sliding engagement contact with, the corresponding runnel (106, 107) provided on the distal elongated outer body component (2b), when the slidable carriage assembly is in the armed position, in order to continue to prevent rotation of the carriage assembly (27) holding the pen injection system, about the central axis (6).

The slidable carriage assembly (27) further comprises a releasable trigger means (111) having a trigger member (112), configured to retain the slidable carriage assembly (27) in the armed position until the trigger member (112) is released. The releasable trigger member (112) is provided to enable the user to effect an injection, and release of the trigger causes the pen injection system (7) to move in a distal direction from the armed position through to an injection position in which the needle of the pen injection system penetrates an injection surface such as the skin. The impetus for moving the pen injection system (7) from the armed position to the unarmed position via an injection operation, is provided by a biasing element (21), such as, for example, a compressed spring. The slidable carriage assembly is thus moved into the armed position, after rotationally activated locking of the pen injection system as described elsewhere in the present specification, by the user of the inserter device (1) pulling or exerting traction, on the pen injection system (7) as held in the slidable carriage assembly (27) by the compression ring (26) and compressible membrane (87), said traction being exerted in a proximal direction. In essence, the user of the automatic needle inserter pulls the pen body backwards, in a proximal direction, against the biasing element (21), compressing the latter, until the trigger member (112) of the releasable trigger is latched, and the armed position set. The trigger member (112) comprises an elastically deformable arm that is movable out of a first plane of longitudinal axial alignment, in the unarmed position, into a second and different plane of longitudinal axial alignment, in the armed position. The elastically deformable arm (112) extends radially outwards from the proximal body part (72) of the carriage assembly (27), and in a proximal direction. The elastically deformable arm (112) lies along a first longitudinal plane, which is in parallel to the central longitudinal axis (6) in the unarmed state, and before deformation. As the slidable carriage assembly (27) is moved in a proximal direction, the elastically deformable arm (112) meets an abutment (113) provided on, and adjacent to, the proximal extremity of the proximal outer body component (2a). FIG. 3B shows a cutaway view of the position of the elastically deformable arm (112) engaged in the armed position. The abutment (113), which lies in the same longitudinal plane as the elastically deformable trigger arm (112), causes the arm to deform elastically and be moved out of the first longitudinal plane into a second longitudinal plane which is radially separate from the first longitudinal plane. In the embodiment as exemplified herein, for example, and as illustrated in FIG. 3B, the trigger arm (112) is elastically deformed out of the first longitudinal plane via a suitably shaped sloping projection (113), extending into, and defining the proximal extremity of a cut out channel (114) provided in the inwardly facing surface of the proximal outer body component (2a). The cutout (114) defines the sloping projection abutment (113) which is further provided with a proximal hook end (115), which interacts and engages with a corresponding and complementary hook end (116) provided on the trigger arm (112). As the trigger arm is elastically deformed out of the first longitudinal plane into the second longitudinal plane by the sloping projection abutment (113), so the hook end (116) of the trigger arm (112) is moved around the hook end (115) of the abutment, and then further movement in the proximal direction causes the hook end (116) of the trigger arm (112) to catch on the hook end (115) of the sloping projection abutment (113). Once this position has been reached, the inserter device is in the armed position, until the trigger is released.

The trigger release is provided by the release button (15) located on the proximal outer body component (2a), and in the exemplified embodiment of the figures, is located on the closure cap (9), but could also be directly integrated by suitable moulding, for example, of the proximal outer body component (2a). The release button (15) comprises an elastically deformable tongue portion (117) which extends in a distal direction and is located over the trigger arm (112). The release button (15) also comprises an inspection orifice (118), which is located over the proximal end of the trigger arm, and enables a user to verify at a glance, that the trigger arm is correctly in place, and thus that the inserter device is correctly armed. Such visualization can be facilitated, for example, by providing the trigger arm with a color that is visible through the inspection orifice (118). The elastically deformable tongue (117) of the release button lies substantially flush with the outside surface of the proximal outer body component, but is deformable via a downward press effected by the user, when it is desired to activate the release of the inserter device (1) and send the slidable carriage assembly in a distal direction to penetrate the target site for injection with the previously mounted and exposed needle. Downward pressing of the tongue (117) therefore causes an inward facing surface of the tongue (117) to come into contact with the proximal end of the trigger arm (112), and move the elastically deformable arm (112) out of the second plane of longitudinal axial alignment back into the first plane of longitudinal axial alignment, due to the arm regaining its normal elastic constraint at rest, and thereby release the trigger member (112) from the armed position. The trigger member (112) is now free to slide back down the cutout channel (114) under the impetus of the detent energy stored in the compressed spring (21) as said spring expands once again to its unconstrained position.

Turning back once more to the slidable carriage assembly (27), said assembly is provided with surface engagement means which comprise at least one projecting contact member (FIG. 3A, 4A, 119) extending radially outwardly from the slidable carriage assembly, and in a proximal direction beyond the proximal extremity of the proximal part (72) of the slidable carriage body. This contact member (119) engages in axial sliding contact with at least one of the corresponding runnels or grooves described elsewhere in the present specification, and provided on the proximal outer body component (2a). Said runnel is dimensioned and configured to surround the projecting contact member (119) and thereby help prevent rotation of the carriage assembly about the central longitudinal axis (6), as the carriage assembly (27) translates from the unarmed position to the armed position, and vice-versa. The contact member (119) can advantageously be represented as a series of radially spaced and outwardly projecting legs (119), positioned on an outwardly facing surface of the first, proximal part (72) of the carriage body, and extending along said carriage body in parallel to the central longitudinal axis (6) beyond the proximal end of the carriage body. The length of the projecting legs extending beyond the proximal extremity of the carriage body is configured to provide an appropriate abutting distance of the proximal end of the carriage body with the proximal end (3) of the outer elongate body (2a, 2b), and thereby limit the distance of travel of the carriage body in the proximal direction. Additionally, said contact member legs are dimensioned and shaped to engage with a distal extremity of the biasing spring (21), pushing against the spring (21) as the slidable carriage assembly is moved from the unarmed position to the armed position, and serving as the contact surface for driving the slidable carriage assembly in the reverse direction, when the release button of the needle inserter device is activated.

A brief description of use of the automatic needle inserter device (1) will now be provided. The pen distal extremity abutment lever arm (23) is manipulated via its prehensile end (24) to cause the lever arm to rotate about the its axis of rotation and move the abutting end (25) of the lever arm into the central longitudinal bore (5). A pen injection system is inserted through opening (12) in the closure cap (9) into the bore (5) of the elongated outer body (2a, 2b) comprising the assembled proximal (2a) and distal (2b) outer body components. The needle mount surface of the needle mount provided on the injection pen comes into abutting contact with the abutting end (25) of the abutment lever arm (23). The abutting end (25) remains in place in the bore (5) until rotational locking of the body of the injection pen (7) has been carried out. Rotationally activated locking occurs by rotating the proximal outer body component around the central longitudinal axis (6), thereby driving rotation of the compression ring (26) and proximal part (72) of slidable carriage assembly (27). Rotation of the compression ring (26) correspondingly compresses the compressible membrane (87) onto the body of the injection pen (7). The compression exerted on the body of the injection pen locks the pen body (7) into an axial position within the bore (5). This position is indicated to the user via the corresponding indicia or visual markings (A, B, C, D, E) provided on the outside surface of the proximal elongated body component (2a). At this stage, although rotationally locked, the needle inserter device is still in the unarmed position. The lever arm (23) is moved back to its position flush with the outer and respectively inner surfaces of the distal outer body component (2b) so that the abutting end (25) no longer projects into the bore (5) of the inserter. The user then removes the needle guard, exposing the injection needle. The compression exerted on the body of the injection pen (7) by the compression ring (26) and compressible membrane is sufficient to enable the pen body to be grasped in one hand, keeping the other hand on the elongated outer body (2a, 2b), and then the pen body (7) is moved in a proximal direction to arm the automatic needle inserter. The pen injector (7) translates in the proximal direction with the slidable carriage assembly (27) and compression ring (26) until the trigger member (112) is deflected by the sloping abutment projection defined by the cutout channel (114) out of its initial longitudinal plane into the second longitudinal plane, and the corresponding hooks (115, 116) engage with each other to set the inserter device (1) in the armed position. The user applies the distal end (4) and distal facing surface (22) to the site of intended injection. Pressing on the tongue (117) of the release button (15) moves the trigger member (112) once more out of the second longitudinal plane back into the first longitudinal plane, releasing the hooks (115, 116) one from the other, and allowing the trigger member, slidable carriage assembly (27) and compression ring (26) to be propelled in a distal direction, under the impetus of the released energy that was stored in the compression spring (21). The propulsion imparted by the spring causes the injection pen (7), and needle mounted thereon, to protrude beyond the distal extremity (4) of the inserter device and into the injection site, at precisely the correct depth, due to the previous axial locking and axial abutment of the injection pen. At this stage, the user can effect injection of the substance to be injected by pressing on the activation button of the injection pen (7) in the usual manner. Once injection has been completed, the automatic needle inserter device (1) can be removed from the injection site, and the rotational locking means unlocked, by rotating the proximal outer body component (2a) about the central axis (6) in the reverse direction to that of the locking movement. This releases the compression exerted by the compression ring (26) on the compressible membrane (87) and frees the pen injection system (7) from its locked axial position. The injection pen (7) can then be removed and/or exchanged as required for further subsequent injections.

1. Automatic needle inserter for a pen injection system comprising:
an elongated inserter body having a proximal extremity and a distal extremity, and a longitudinal bore extending through the elongate body from the proximal extremity to the distal extremity, the longitudinal bore having a central longitudinal axis, wherein the elongated inserter body is dimensioned and configured to receive a pen injection system introduced into said bore via the proximal extremity of the inserter body, and is further configured and dimensioned to prevent the pen injection system from exiting the longitudinal bore via the distal extremity;
the elongated inserter body further comprising:
a rotationally-activated locking means configured to lock the pen injection system in an axial position within the longitudinal bore through rotation of at least a part of the elongated inserter body about the central longitudinal axis from a first, non-locking position to at least one or more second, locking positions.

2. Automatic needle inserter according to claim 1, wherein the elongated inserter body comprises at least a first elongated outer body component, and at least a second elongated outer body component, and wherein at least one of the at least first elongated outer body component and the at least second elongated body outer component is configured to rotate during locking about the central longitudinal axis with respect to the at least other elongated outer body component.

3. Automatic needle inserter according to claim 1, wherein the elongated inserter body comprises at least a first elongated outer body component, and at least a second elongated outer body component, and wherein the at least first elongated outer body component and at the least second elongated outer body component, are configured to rotate during locking about the central longitudinal axis in opposite directions, one with respect to the other.

4. Automatic needle inserter according to claim 1, wherein the rotationally-activated locking means comprises a compression ring having a nominal thickness, a nominal internal diameter defining a central bore, and wherein the compression ring is coaxially located with the longitudinal bore of the elongated inserter body.

5. Automatic needle inserter according to claim 4, wherein the compression ring has a variable internal diameter with respect to the nominal internal diameter.

6. Automatic needle inserter according to any claim 4, wherein the compression ring comprises at least one portion having an increased internal diameter with respect to the nominal diameter.

7. Automatic needle inserter according to claim 4, wherein the compression ring comprises at least one portion having a reduced internal diameter with respect to the nominal diameter.

8. Automatic needle inserter according to claim 4, wherein the at least one portion of the compression ring having an increased internal diameter extends around the circumference of the internal diameter of the compression ring with a gradual reduction of internal diameter towards a portion of the compression ring having the reduced internal diameter.

9. Automatic needle inserter according to claim 4, wherein the compression ring comprises at least one portion having an increased thickness with respect to a nominal thickness of the ring, extending inwardly into a central bore.

10. Automatic needle inserter according to claim 4, wherein the compression ring comprises at least one portion having a reduced thickness with respect to a nominal thickness of the ring, extending inwardly into a central bore.

11. Automatic needle inserter according to claim 4, wherein the at least one portion of the compression ring having an increased thickness extends around from an outer periphery of the compression ring inwardly into a central bore with a gradual reduction of thickness towards a portion of the compression ring having the reduced thickness.

12. Automatic needle inserter according to claim 1, wherein the rotationally-activated locking means further comprises a compressible membrane of resilient material.

13. Automatic needle inserter according to claim 4, wherein the compressible membrane of resilient material is coaxially located within the bore of the compression ring.

14. Automatic needle inserter according to claim 13, wherein the compressible membrane is compressed from a relaxed state to a compressed state via rotational movement of the compression ring about the central longitudinal axis from the first, non-locking position to the one or more second, locking positions.

15. Automatic needle inserter according to claim 14, wherein an inwardly facing surface of the compressible membrane is brought into contact with an outside surface of a body of the pen injection system via rotational movement of the compression ring about the central longitudinal axis from the first, non-locking position to the one or more second, locking positions.

16. Automatic needle inserter according to claim 4, wherein the compression ring is attached to, or mounted on, a slidable carriage assembly configured to translate the compression ring along the central longitudinal axis from an unarmed position to an armed position.

17. Automatic needle inserter according to claim 16, wherein the slidable carriage assembly comprises a bore which is coaxially aligned with the bore of the compression ring.

18. Automatic needle inserter according to claim 16, wherein at least a part of the slidable carriage assembly is configured to co-rotate with the compression ring about the central longitudinal axis.

19. Automatic needle inserter according to claim 16, wherein the slidable carriage assembly and compression ring each comprise surface engagement means configured to engage in sliding engagement with the first elongated outer body component, to enable translation of the compression ring, along with the slidable assembly, from the unarmed position to the armed position.

20. Automatic needle inserter according to claim 19, wherein the surface engagement means of the slidable carriage assembly comprise at least one projecting contact member extending radially outwardly from the slidable carriage assembly.

21. Automatic needle inserter according to claim wherein the at least one projecting contact member extending radially outwardly from the slidable carriage assembly engages in axial sliding contact with at least one corresponding runnel provided on the first elongated outer body component.

22. Automatic needle inserter according to claim 20, wherein the at least one projecting contact member extending radially outwardly from the slidable carriage assembly also extends proximally beyond a proximal extremity of the slidable carriage assembly.

23. Automatic needle inserter according to claim 16, wherein the slidable carriage assembly comprises at least one slider arm extending distally from a distal part of the carriage assembly in parallel to the central longitudinal axis.

24. Automatic needle inserter according to claim 23, wherein the at least one slider arm engages in sliding axial contact with at least one corresponding runnel provided on the second elongated outer body component.

25. Automatic needle inserter according to claim 24, wherein the at least one slider arm has a length sufficient to extend distally into, and maintain sliding engagement contact with, the corresponding runnel provided on the second elongated outer body component, when the slidable carriage assembly is in the armed position.

26. Automatic needle inserter according to claim 19, wherein the compression ring comprises at least one projecting contact member extending radially outwardly from the compression ring.

27. Automatic needle inserter according to claim 26, wherein the at least one projecting contact member extending radially outwardly from the compression ring engages in axial sliding contact with at least one corresponding runnel provided on the first elongated outer body component.

28. Automatic needle inserter according to claim 16, wherein the slidable carriage assembly further comprises a releasable trigger means having a trigger member, configured to retain the slidable carriage assembly in the armed position until the trigger member is released.

29. Automatic needle inserter according to claim 28, wherein the trigger member comprises an elastically deformable arm that is movable out of a first plane of longitudinal axial alignment in the unarmed position into a second plane of longitudinal axial alignment in the armed position.

30. Automatic needle inserter according to claim 29, wherein the first elongated outer body component comprises a release button configured to move the elastically deformable arm out of the second plane of longitudinal axial alignment into the first plane of longitudinal axial alignment, and thereby release the trigger member from the armed position.

31. Automatic needle inserter according to claim 1, further comprising a selectively actionable pen distal extremity abutment means configured to abut a distal extremity of the injection pen system, upon insertion of the pen injection system into the longitudinal bore of the elongated inserter body, along the central longitudinal axis of the longitudinal bore.

32. Automatic needle inserter according to claim 31, wherein the selectively actionable pen distal extremity abutment means is movable from a first, non-abutting position, to a second, abutting position.

33. Automatic needle inserter according to claim 31, wherein the selectively actionable pen distal extremity abutment means is located adjacent, or in proximity, to the distal extremity of the elongated inserter body.

34. Automatic needle inserter according to claim 31, wherein the selectively actionable pen distal extremity abutment means comprises an articulated arm member, configured to rotate about an axis in parallel alignment to the central longitudinal axis, and wherein the articulated arm member is rotatable about said parallel axis of rotation from the first, non-abutting position, to the second, abutting position.

35. Automatic needle inserter according to claim 34, wherein the articulated arm member lies flush with the elongated inserter body in the first, non-abutting position, and extends into the longitudinal bore in the second, abutting position.

36. Automatic needle inserter according to claim 31 wherein the rotationally activated locking of the pen injection system occurs only after abutting of the pen injection system via selective actioning of the abutment means.
